(19) United States
(12) Patent Application Publication
(10) Pub. No.: US 2009/0241945 A1
(43) Pub. Date: Oct. 1, 2009

(54) DEVICE FOR DELIVERING A RESPIRATORY GAS

(76) Inventor: Rainer Müllner, Osterreich (AT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/281,044

(22) PCT Filed: Feb. 27, 2005
(86) PCT No.: PCT/EP2007/001681

§ 371 Date: Dec. 19, 2008

(30) Foreign Application Priority Data
Mar. 1, 2006 (DE) 10 2006 009 329.1

(51) Int. Cl. A61M 16/00

(52) U.S. Cl. 128/202.22

(57) ABSTRACT

A device is described for making available a respiratory gas (mixture) to a patient, comprising means for making available one or more gases and means for delivering the respiratory gas (mixture) to the patient, their being assigned a respiratory air line that is connected to the surrounding area and in which a negative pressure valve is arranged. According to the invention, the negative pressure valve (D) is dynamically connected to a device for generating a warning signal (C, E), preferably an acoustic and/or optical warning signal.

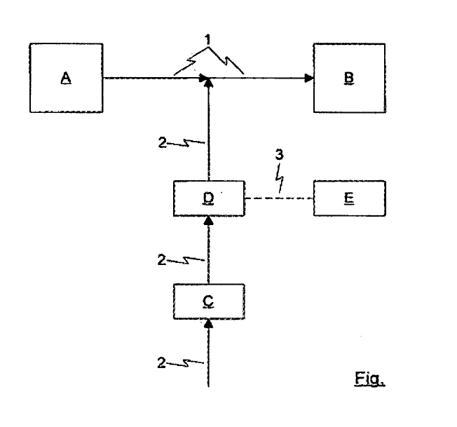

The invention relates to a device for making available a respiratory gas (mixture) to a patient, comprising means for making available one or more gases and means for delivering the respiratory gas (mixture) to the patient, their being assigned a respiratory air line that is connected to the surrounding area and in which a negative pressure valve is arranged.

Generic devices for making available a respiratory gas (mixture) to a patient are well known from the prior art. They are used to deliver a respiratory or inhalation gas (mixture) to a patient.

In such devices, safety devices must be provided that enable the patient to continue to breathe in the case of equipment failure or gas supply failure. In order to be able to ensure this, to date means for delivering a respiratory gas (mixture) to a patient have been assigned a respiratory air line that is connected to the surrounding area and in which a negative pressure valve is arranged.

Providing this respiratory air line and the negative pressure valve ensures that even in the case of equipment failure or of failure of the gas supply, the patient can continue to breathe. The negative pressure that is necessary for opening the negative pressure valve is generated in this connection by the patient himself. After opening the negative pressure valve, the patient can inhale ambient air via the respiratory air line.

In the known generic devices for making available a respiratory gas (mixture) to a patient, there is, however, the disadvantage that the medical or attendant personnel are not alerted as soon as the patient activates the negative pressure valve.

The object of this invention is to devise a generic device for making available a respiratory gas (mixture) for a patient that avoids the aforementioned disadvantages and ensures reliable alerting of the attendant personnel in the case of equipment failure or in a failure of the gas supply.

To achieve the aforementioned object, a generic device for making available a respiratory gas (mixture) to a patient is proposed that is characterized in that the negative pressure valve is dynamically connected to a device for generating a warning signal, preferably an acoustic and/or optical warning signal.

Other advantageous configurations of the device according to the invention for making available a respiratory gas (mixture) to a patient are characterized in that
the device for generating a warning signal is made as a vibration generator that is located in the respiratory air line,
the threshold value of the negative pressure valve is adjustable, and
the vibration generator with respect to its air flow is made adjustable.

Since, according to the invention, the negative pressure valve at this point is dynamically connected to a device for generating a warning signal, preferably an acoustic and/or optical warning signal, in the event of interruption of the delivery of the respiratory or inhalation gas (mixture), the medical or attendant personnel can be immediately notified thereof and can initiate the required measures—replacement or repair of the defective unit.

According to one advantageous configuration of the device according to the invention for making available a respiratory gas (mixture) to a patient, the device for generating a warning signal is preferably designed as a vibration generator located in the respiratory air line. In this connection, for example, a whistle through which the respiratory air taken in by the patient flows can be used as the vibration generator.

The device according to the invention for making available a respiratory gas (mixture) to a patient and other advantageous configurations thereof will be explained in more detail below using the embodiment shown in the FIGURE.

The FIGURE shows in schematic form a device for making available a respiratory gas (mixture) to a patient B. The latter comprises means for making available one or more gases—shown by the black box A in the FIGURE—and means for delivering the respiratory gas (mixture) to the patient—in the FIGURE shown by the line 1. The means for making available one or more gases A have at least mixing and/or metering means for the gas or gases to be delivered to the patient B.

During regular operation, delivery of the respiratory or inhalation gas (mixture) takes place from the means for making available one or more gases A to the patient B via the line 1. The negative pressure valve D to be provided in the line 2 is closed, so that no ambient air or inside air is delivered to the patient B via the line 2.

If, at this point, some equipment failure and an associated failure of the gas supply via line 1 to the patient B takes place, the negative pressure valve D is activated by the negative pressure produced by the patient B and is opened so that ambient air or inside air can be delivered to the patient B via the line 2.

In this connection, it need not necessarily be complete failure of the device according to the invention for making available a respiratory gas (mixture); rather, even in the case of inadequate delivery of the respiratory or inhalation gas (mixture) via the line 1 to the patient B, a delivery of ambient air or inside air via the line 2 can take place by the negative pressure thereby generated.

To further develop the device according to the invention for making available a respiratory gas (mixture), it is suggested that the threshold value of the negative pressure valve D, i.e., the pressure value at which the negative pressure valve D opens, be adjustable.

According to the invention, the negative pressure valve D at this point is dynamically connected to a device for producing a warning signal—shown by the black box E that is connected to the negative pressure valve D via the line 3 that is shown by the broken line—so that prompt and immediate alerting of medical personnel can be accomplished. In addition to a host of other possibilities, the negative pressure valve D is preferably dynamically connected to a device for generating an acoustic and/or optical warning signal.

If the device for generating a warning signal is made as a vibration generator C that is located in the respiratory air line—and with the negative pressure valve D opened—the ambient or inside air flows through the vibration generator C, which results in an acoustic signal that alerts the medical or attendant personnel. Activation of the alarm therefore takes place exclusively during inhalation by the patient.

In this connection, according to one advantageous configuration of the device according to the invention—the vibration generator C can be made adjustable with respect to its air flow. The alert threshold can be indirectly set by means of this configuration.

1. In a device for making available a respiratory gas to a patient, comprising a first conduit for delivering the respiratory gas to the patient, a second conduit for delivering emergency respiratory air to the patient and being in on-off communication with said first conduit, and a negative pressure valve being integrated in said second conduit, the improvement wherein the negative pressure valve (D) is dynamically connected to a device for generating a warning signal (C, E), said warning signal being activatable when the patient inhales via the respiratory air line (2).

2. A device according to claim 1, wherein the device for generating a warning signal comprises a vibration generator (C) located in the respiratory air line (2).

3. A device according to claim 2, said negative pressure valve (D) having an adjustable threshold value.

4. A device according to claim 2, wherein the vibration generator (C) is adjustable in response to air flow.

5. A device according to claim 3, wherein the vibration generator (C) in response to air flow.

6. A device according to claim 1, wherein said warning signal is acoustic and/or optical.
